(19) United States
(12) Patent Application Publication
(10) Pub. No.: US 2016/0089232 A1
(43) Pub. Date: Mar. 31, 2016

(54) SYSTEMS, INTRAOCULAR LENSES, AND METHODS FOR TREATMENT OF POSTERIOR CAPSULE OPACIFICATION

(71) Applicants: Charles DeBoer, Sierra Madre, CA (US); Ramiro Magalhaes Ribeiro, South Pasadena, CA (US); Mark S. Humayun, Glendale, CA (US); Yu-Chong Tai, Pasadena, CA (US); Sean Caffey, Pasadena, CA (US)

(72) Inventors: Charles DeBoer, Sierra Madre, CA (US); Ramiro Magalhaes Ribeiro, South Pasadena, CA (US); Mark S. Humayun, Glendale, CA (US); Yu-Chong Tai, Pasadena, CA (US); Sean Caffey, Pasadena, CA (US)

(21) Appl. No.: 14/867,054

(22) Filed: Sep. 28, 2015

Related U.S. Application Data
(60) Provisional application No. 62/056,950, filed on Sep. 29, 2014.
(60) Provisional application No. 62/066,134, filed on Oct. 20, 2014.

(51) Int. Cl. A61F 2/16; A61F 9/007; A61F 9/008

(52) U.S. Cl. CPC A61F 2/16015; A61F 9/00802; A61F 9/00745; A61F 2002/16901; A61F 2009/0087; A61F 2009/00887

(57) ABSTRACT

In various embodiments, ocular treatment systems, intraocular lenses, and treatment methods are utilized for the treatment or prevention of posterior capsule opacification of the eye by, for example, targeting portions of the posterior lens capsule for ablation while minimizing damage to implanted intraocular lenses.

### RELATED APPLICATIONS

This application claims the benefit of and priority to U.S. Provisional Patent Application No. 62/056,950, filed Sep. 29, 2014, and U.S. Provisional Patent Application No. 62/066,134, filed Oct. 20, 2014, the entire disclosure of each of which is hereby incorporated herein by reference.

### TECHNICAL FIELD

In various embodiments, the present invention relates to intraocular lens designs and methods of treatment for posterior capsule opacification.

### BACKGROUND

The crystalline lens of a human's eye refracts and focuses light onto the retina. Normally the lens is clear, but it can become opaque (i.e., when developing a cataract) due to aging, trauma, inflammation, metabolic or nutritional disorders, or radiation. While some lens opacities are small and require no treatment, others may be large enough to block significant fractions of light and decrease the visual acuity. In such cases, surgical removal of the cataract is often necessary.

Conventionally, cataract treatments involve surgically removing the opaque lens matrix from the lens capsule using, for example, manual extraction and aspiration, phacoemulsification, and/or application of a femtosecond laser through a corneal incision. An artificial intraocular lens (IOL, or simply “lens”) may then be implanted in the lens capsule bag to replace the crystalline lens (see, e.g., U.S. patent application Ser. No. 14/058,634, filed Oct. 21, 2013, the entire disclosure of which is incorporated by reference herein).

Generally, IOLs are made of a foldable, optically transparent polymeric material, such as silicone or acrylic, for minimizing the incision size and required stitches and, as a result, the patient's recovery time. The most commonly used IOLs are single-element lenses (or monofocal IOLs, non-accommodating IOLs, or non-focusing IOLs) that provide a single focal distance for distance vision. Typically, distance vision requires limited contraction of ciliary muscles in the eye (i.e., emmetropia); thus monofocal IOL designs are relatively simple. For example, to choose an appropriate geometry for a monofocal lens having a desired focusing power, limiting factors of the eye's anatomy, such as the axial eye length and the power of the cornea, are taken into consideration. However, because the focal distance is not adjustable following implantation of the monofocal IOL, patients implanted with such lenses can no longer focus on objects at a close distance (e.g., less than twenty-five centimeters); this results in poor visual acuity at close distances.

In some patients, after the implantation of the IOL, the posterior part of the lens capsule becomes opaque (due to, e.g., overgrowth of epithelium cells), triggering a condition called posterior capsule opacification (PCO). PCO decreases the amount of light that reaches the retina, causing a decrease in visual acuity. PCO is one of the most common complications after cataract extraction. Approximately 30-40% of the subjects who undergo cataract extraction and IOL implantation develop PCO and require treatment.

FIG. 1A depicts the structure of a human eye 100 after implantation of an IOL 105. As shown, the IOL 105 fits within the lens capsule 110, which is held in place by connective tissue 115 (e.g., natural lens ligaments or zonules) behind the iris 120 and the cornea 125, and in front of the retina 130. The volume between the retina 130 and the lens capsule 110 is typically filled with vitreous humor, and the anterior chamber between the lens capsule 110 and the cornea 125 is typically filled with aqueous humor. In FIG. 1A, PCO has resulted in formation of an opaque region 135 on the posterior portion of the lens capsule. Currently, treatment of PCO is performed by breaking the posterior capsule using a Nd:YAG (neodymium-doped yttrium aluminum garnet; Nd:Y₃Al₅O₁₂) laser. When applied to the posterior capsule, the laser energy produces thermal and mechanical damage thereto. The laser energy may rupture the posterior capsule, thereby removing the opaque region of the lens capsule from the central visual axis. FIG. 1B depicts the eye 100 after application of laser energy and resultant formation of a hole 140 in the lens capsule 110 that removes all or a portion of the opacity from the visual axis.

While such laser-based treatments have a reasonably high success rate, in some cases, the laser beam may damage the IOL, leading to a decrease in visual acuity and contrast and causing glare for the patient. Such damage may occur due to incorrect focusing of the laser energy. In some cases, however, the IOL is sufficiently close to the posterior capsule that even laser light correctly focused on the posterior capsule may damage the IOL. Such damage may range in severity from mild to moderate, and even severe. Mild damage may be characterized as small pits with raised smooth edges, while moderate damage may include deeper pits and/or large craters or small cracks, and severe damage may include severe cracking or crevice formation into the optic.

Recently, accommodating intraocular lenses (AIOLs) have been developed to provide adjustable focal distances (or accommodations) by relying on the natural focusing ability of the eye. Because an AIOL works closely in coordination with the eye tissue, the focusing power thereof is sensitive to the geometric properties of the eye tissue. For example, ciliary muscles of the eye may contract, causing a change in the diameter and/or the shape of the lens capsule accommodating the AIOL; this results in an adjustment of the focal distance. Additionally, the shorter the focal distance of the AIOL, the more sensitive the AIOL will be to a change in geometry of the eye tissue.

Most IOLs are made of single piece of hard material, although some newer IOLs have a two-lens design, and lenses filled with clear fluid have also been utilized. Most current IOLs are prefabricated for their lens power and then placed in the eye, but again, a few designs involve intraocular filling of the liquid in the lens at the time of initial surgery or possibly at a subsequent time (e.g., for adjustment or should the liquid become opacified, or even simply to exchange the liquid in the lens for a liquid of different properties (e.g., optical, viscosity, color)). A liquid-filled bag that provides accommodation—made from, for example, an elastic, biocompatible polymer—results in numerous benefits and advantages, e.g., the ability to adjust the lens following implantation; to customize the lens to the needs of each patient; to accommodate vision; sharper vision over a wide range of distances; and reduction of visual side effects such as glares and halos. See, e.g., U.S. Pat. No. 8,771,347, and U.S. patent application Ser. No. 13/473,012, filed May 16, 2012, the entire disclosure of each of which is hereby incorporated by reference.

Liquid-filled IOLs impose particular challenges for PCO treatments that open the posterior lens capsule. With liquid-filled IOLs, the laser energy may cause a rupture of the surrounding shell of the IOL, causing the liquid components to leak out and degradation of optical performance. For liquid-filled AIOLs, this may even damage the mechanism of accommodation.

In view of the foregoing, there is a need for IOL designs and treatment techniques that minimize or substantially eliminate complications resulting from PCO treatments.

### SUMMARY

In accordance with various embodiments of the present invention, PCO treatment techniques are modified to prevent or substantially eliminate damage to IOLs, and/or IOLs are designed to more robustly resist damage from PCO treatments. Various embodiments of the present invention may be used with any type of IOL, including conventional IOLs and liquid-filled IOLs.

Embodiments of the present invention treat or prevent PCO by opening the posterior lens capsule prior to (or concomitant with) IOL implantation, or moving the IOL away from the posterior lens capsule (i.e., posteriorly and/or equatorially) prior to and during the opening procedure. While embodiments of the invention involve removal of the posterior portion of the lens capsule after PCO formation, they may also be used preventatively to open an aperture in the posterior lens capsule before PCO formation. In this manner, the optical axis of the eye never has a degradation of visualization due to PCO formation, as cells cannot migrate to the optical axis.

In other embodiments of the invention, the posterior lens capsule is cut in a specific, perforated pattern before IOL implantation in order to reduce or substantially minimize the amount of, e.g., cutting or laser or ultrasound energy utilized to open the posterior lens capsule. Such embodiments may also be combined with IOL movement and/or spacing away from the posterior lens capsule. Patterning of the posterior lens capsule may be implemented in tandem with IOLs having markings indicating still-intact portions of the capsule requiring removal for complete opening of the capsule. The markings may be accompanied by, or may include or consist essentially of, strengthened and/or thickened portions of the IOL that are more resistant to the effects of laser treatments for opening the lens capsule. A visible-, optical-, or ultrasound-guided laser may be used to detect the markings (e.g., two or more points) in order to determine IOL position, position relative to the capsule, and appropriate areas in the sagittal plane to cut the lens capsule.

Techniques utilizable to cut the capsule in accordance with embodiments of the present invention include mechanical or laser removal (e.g., conventional Nd:YAG, femtosecond, nanosecond, excimer, diode, argon, attosecond, etc. lasers) or ultrasound sources. Measurement or guidance systems may be used with the cutting systems during the procedure (i.e., simultaneous image (real-time) systems), or such systems may be used to ensure appropriate spacing before the procedure. Examples of measurement or guidance systems include optical coherence tomography, ultrasound, ultrasound biomicroscopy, scheimpflug imaging, tomography, magnetic resonance imaging, aberrometery, and slit lamp illumination. In various embodiments, a guidance system and a posterior capsular opening system are used together. For example, OCT may be used to guide a femtosecond laser and plan the cut path of the posterior lens capsule.

In an aspect, embodiments of the invention feature a method of treating posterior capsule opacification (PCO) in a patient's eye comprising (i) a lens capsule having a posterior surface exhibiting (or at risk for) PCO, and (ii) an intraocular lens implanted within the lens capsule. The intraocular lens is urged away from the posterior surface of the lens capsule so that a distance between the intraocular lens and the lens capsule is greater than a threshold distance. Thereafter, at least a portion of the posterior surface of the lens capsule is removed.

Embodiments of the invention may include one or more of the following in any of a variety of combinations. The threshold distance may be approximately 10 μm, approximately 500 μm, or even approximately 3000 μm. Removing the at least a portion of the posterior surface of the lens capsule may include or consist essentially of focusing laser or ultrasound energy thereon. Urging the intraocular lens away from the posterior surface of the lens capsule may include or consist essentially of introducing a fluid (i.e., a liquid and/or a gas) between the intraocular lens and the posterior surface of the lens capsule. The viscosity of the fluid may be at least 1 centistoke, or even at least 1000 centistokes. The intraocular lens may include (i) a peripheral edge at least a portion of which is sealed against the lens capsule, and (ii) a posterior optical surface in opposed relation to the posterior surface of the lens capsule to form a compartment including both of the surfaces. The intraocular lens may be an inflatable intraocular lens. Urging the intraocular lens away from the posterior surface of the lens capsule may include or consist essentially of removing fluid (i.e., liquid and/or gas) from at least a portion (e.g., at least one of one or a plurality of compartments) of the intraocular lens.

In another aspect, embodiments of the invention feature an intraocular lens for implantation into a lens capsule of an eye. The intraocular lens includes or consists essentially of a lens portion for replacement of a natural lens of the eye, and a sealing portion configured to sealingly contact the lens capsule to form a sealed compartment between the intraocular lens and a posterior surface of the lens capsule. The sealing portion is disposed peripherally to the lens portion. The sealing portion may be pierceable and/or moveable to allow introduction of fluid between the intraocular lens and the posterior surface of the lens capsule.

In yet another aspect, embodiments of the invention feature a method of treatment of an eye comprising a lens capsule from which a natural lens has been removed. A perforation pattern is formed in a posterior surface of the lens capsule. The perforation pattern includes or consists essentially of (i) one or more perforations extending through at least a portion of a thickness of the lens capsule, and (ii) one or more intact areas proximate the one or more perforations. After formation of the perforation pattern, an intraocular lens is implanted within the lens capsule. After implantation of the intraocular lens, an aperture is formed within the posterior surface of the lens capsule by ablating at least one of the intact areas of the perforation pattern.

Embodiments of the invention may include one or more of the following in any of a variety of combinations. Forming the perforation pattern may include or consist essentially of cutting the posterior surface of the lens capsule mechanically and/or by application of energy to the posterior surface of the lens capsule. Prior to forming the aperture, at least a portion of the intraocular lens may be urged away from the posterior surface of the lens capsule. Urging the at least a portion of the intraocular lens away from the posterior surface of the lens capsule may include or consist essentially of introducing a fluid (i.e., a liquid and/or a gas) between the intraocular lens and the posterior surface of the lens capsule. Forming the aperture may include or consist essentially of urging at least a portion of the intraocular lens against at least one of the intact areas of the perforation pattern. The intraocular lens may include one or more fiducial markings thereon. Implanting the intraocular lens within the lens capsule may include or consist essentially of substantially aligning at least one fiducial marking with an intact area of the perforation pattern. At each fiducial marking, (i) a thickness of the intraocular lens may be greater than a thickness of the intraocular lens away from the fiducial marking, and/or (ii) a coating may be disposed on the intraocular lens. Ablating at least one of the intact areas of the perforation pattern may include or consist essentially of focusing laser or ultrasound energy through at least one of the fiducial markings

In another aspect, embodiments of the invention feature a method of treatment (i) of an eye comprising a lens capsule from which a natural lens has been removed, and (ii) using an intraocular lens (a) having one or more fiducial markings thereon and (b) implantable within the lens capsule with a desired alignment. A perforation pattern is formed in a posterior surface of the lens capsule. The perforation pattern includes or consists essentially of (i) one or more perforations extending through at least a portion of a thickness of the lens capsule, and (ii) one or more intact areas proximate the one or more perforations. After formation of the perforation pattern, an intraocular lens is implanted within the lens capsule with the desired alignment. At least a portion of the perforation pattern is substantially aligned with one or more of the fiducial markings on the intraocular lens. After implantation of the intraocular lens, an aperture is formed within the posterior surface of the lens capsule by ablating at least one of the intact areas of the perforation pattern.

Embodiments of the invention may include one or more of the following in any of a variety of combinations. Forming the perforation pattern may include or consist essentially of cutting the posterior surface of the lens capsule mechanically and/or by application of energy to the posterior surface of the lens capsule. Prior to forming the aperture, at least a portion of the intraocular lens may be urged away from the posterior surface of the lens capsule. Urging the at least a portion of the intraocular lens away from the posterior surface of the lens capsule may include or consist essentially of introducing a fluid (i.e., a liquid and/or a gas) between the intraocular lens and the posterior surface of the lens capsule. Forming the aperture may include or consist essentially of urging at least a portion of the intraocular lens against at least one of the intact areas of the perforation pattern. At each fiducial marking, (i) a thickness of the intraocular lens may be greater than a thickness of the intraocular lens away from the fiducial marking, and/or (ii) a coating may be disposed on the intraocular lens. Ablating at least one of the intact areas of the perforation pattern may include or consist essentially of focusing laser or ultrasound energy through at least one of the fiducial markings

In another aspect, embodiments of the invention feature an intraocular lens for implantation into a lens capsule of an eye. The intraocular lens includes or consists essentially of a lens portion for replacement of a natural lens of the eye, and one or more fiducial markings (e.g., disposed on or in the lens portion or peripherally to the lens portion) for alignment with a perforation pattern disposed within a posterior surface of the lens capsule. At each fiducial marking, (i) a thickness of the intraocular lens may be greater than a thickness of the intraocular lens away from the fiducial marking, and/or (ii) a coating may be disposed on the intraocular lens.

In yet another aspect, embodiments of the invention feature an ocular treatment system usable for the treatment or prevention of posterior capsule opacification of a patient's eye comprising a lens capsule in which an intraocular lens is implanted, the intraocular lens having (A) a peripheral edge at least a portion of which is sealed against the lens capsule, and (B) a posterior optical surface in opposed relation to a posterior surface of the lens capsule to form a compartment including both of the surfaces. The system includes or consists essentially of (i) an imaging system for acquiring images of features within the patient's eye, the features comprising the intraocular lens and a posterior surface of the lens capsule, (ii) a measurement unit for determining relative spacings between features imaged by the imaging system, (iii) a fluid actuation system for introducing fluid into the compartment, the fluid actuation system cooperating with the measurement unit to cause a distance between the posterior optical surface and the posterior surface of the lens capsule to exceed a threshold distance, and (iv) a removal system for removing at least a portion of the posterior surface of the lens capsule.

Embodiments of the invention may include one or more of the following in any of a variety of combinations. The system may include a guidance system for controlling the removal system based on, at least in part, spacings determined by the measurement unit. The guidance system may be configured to recognize fiducial markings on the intraocular lens and control the removal system on the basis thereof. The removal system may include or consist essentially of a mechanical cutter, a laser, an ultrasound system, and/or a fluid removal system.

In another aspect, embodiments of the invention feature a method of treating posterior capsule opacification (PCO) in a patient's eye comprising (i) a lens capsule having a posterior surface exhibiting PCO, and (ii) an intraocular lens implanted within the lens capsule, the intraocular lens having (A) a peripheral edge at least a portion of which is sealed against the lens capsule, and (B) a posterior optical surface in opposed relation to the posterior surface of the lens capsule to form a compartment including both of the surfaces. A liquid is infiltrated into the compartment to urge the intraocular lens away from the posterior surface of the lens capsule so as to increase a distance between the posterior optical surface and the posterior surface of the lens capsule so that it exceeds a threshold distance. Thereafter, at least a portion of the posterior surface of the lens capsule is removed.

Embodiments of the invention may include one or more of the following in any of a variety of combinations. The threshold distance may be approximately 10 μm, approximately 500 μm, or even approximately 3000 μm. Removing the at least a portion of the posterior surface of the lens capsule may include or consist essentially of focusing laser or ultrasound energy thereon. The viscosity of the liquid may be at least 1 centistoke, or even at least 1000 centistokes.

In another aspect, embodiments of the invention feature a method of treating posterior capsule opacification (PCO) in a patient's eye comprising (i) a lens capsule having a posterior surface exhibiting PCO, and (ii) an intraocular lens implanted within the lens capsule. The intraocular lens away is urged from the posterior surface of the lens capsule so that a distance between the intraocular lens and the lens capsule is greater than a threshold distance. Thereafter, at least a portion of the posterior surface of the lens capsule is removed. Urging the intraocular lens away from the posterior surface of the lens capsule may include or consist essentially of infiltrating a liquid into the compartment. The intraocular lens may be an inflatable intraocular lens. Urging the intraocular lens away from the posterior surface of the lens capsule may include or consist essentially of removing fluid from at least a portion of the intraocular lens.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean ±10%, and in some embodiments, ±5%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a schematic cross-section of a human eye with an IOL and experiencing PCO;

FIG. 1B is a schematic cross-section of a human eye after PCO treatment;

FIG. 2A is a schematic view of an IOL implanted within a lens capsule in accordance with embodiments of the invention;

FIG. 2B is a schematic view of an IOL being urged away from the posterior lens capsule during PCO treatment in accordance with embodiments of the invention;

FIG. 2C is a schematic view of the lens capsule and IOL of FIG. 2B after PCO treatment in accordance with embodiments of the invention;

FIG. 3A is a schematic view of an inflatable IOL disposed within a lens capsule in accordance with embodiments of the invention;

FIG. 3B is a schematic view of the IOL of FIG. 3A after partial or substantially full deflation and during PCO treatment in accordance with embodiments of the invention;

FIG. 3C is a schematic view of the IOL and lens capsule of FIGS. 3A and 3B after PCO treatment in accordance with embodiments of the invention;

FIGS. 4A-4D are schematic views of posterior lens capsules having perforated patterns disposed therein in accordance with embodiments of the invention;

FIG. 5A is a schematic view of an inflatable IOL having an aperture-forming feature and implanted within a lens capsule in accordance with embodiments of the invention;

FIG. 5B is a schematic view of the inflatable IOL of FIG. 5A after partial or substantially full inflation in accordance with embodiments of the invention;

FIG. 6A is a schematic view of a posterior lens capsule having a perforated pattern disposed therein in accordance with embodiments of the invention;

FIG. 6B is a schematic view of an IOL having markings for alignment relative to the perforated pattern of FIG. 6A in accordance with embodiments of the invention;

FIG. 7 is a schematic block diagram of an optically guided imaging and ablation system in accordance with embodiments of the invention; and

FIG. 8 is a schematic of an exemplary map of a posterior lens capsule in accordance with embodiments of the invention.

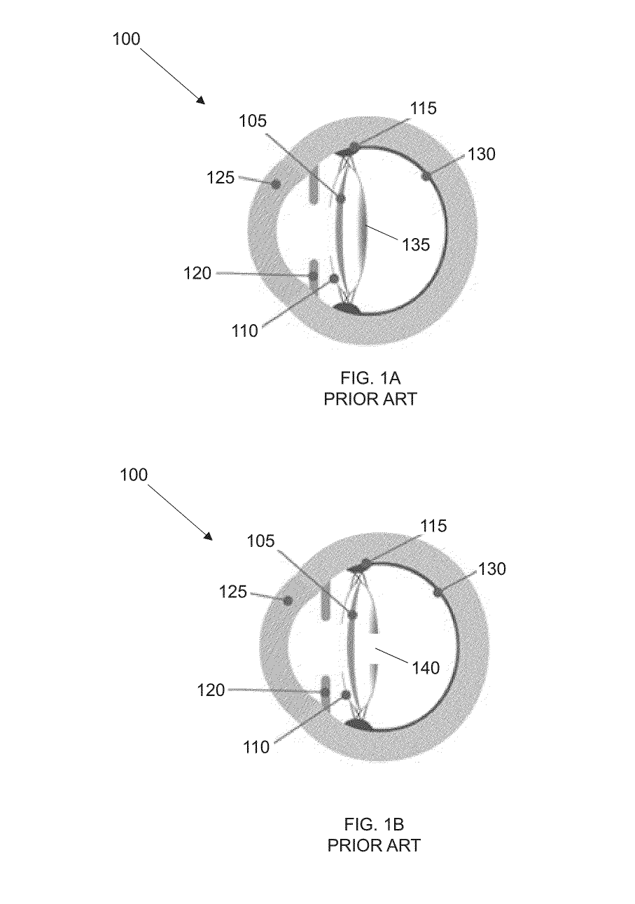

### DETAILED DESCRIPTION

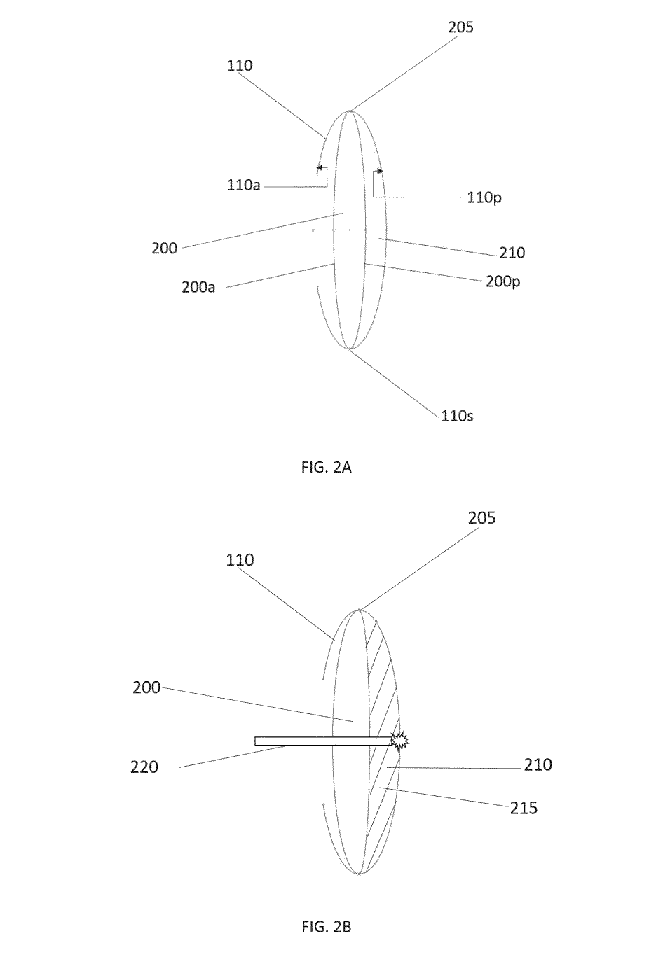

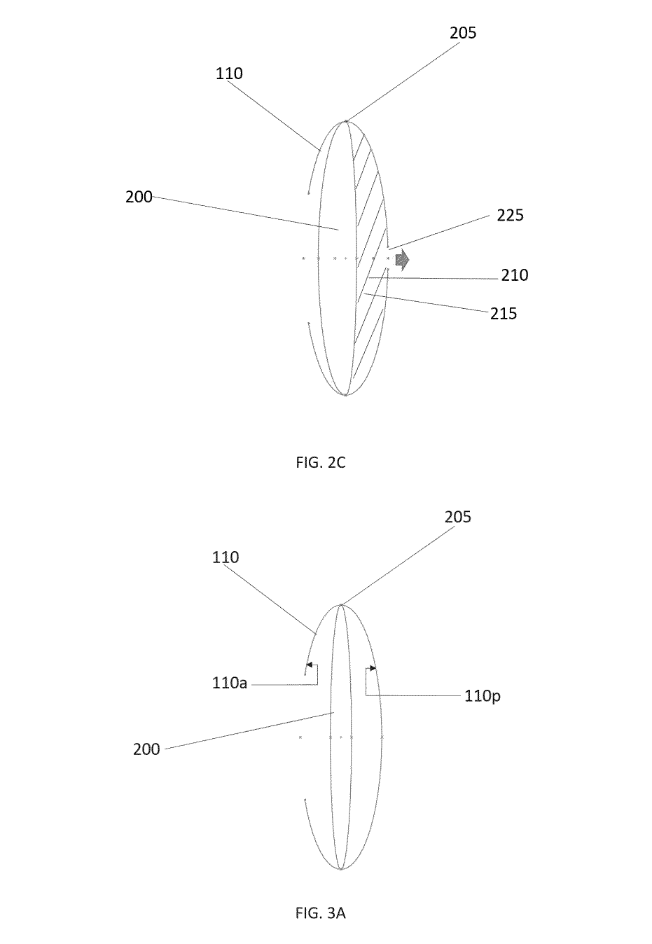

Embodiments of the present invention feature PCO treatments in which the IOL and the posterior lens capsule are spaced apart prior to removing the posterior lens capsule. Refer to FIG. 2A, which depicts a conformally fitting IOL 200 implanted in a lens capsule 110. As shown, the lens capsule 110 has an anterior surface 110a, a posterior surface 110p, and a peripheral surface 110s between anterior surface 110a and posterior surface 110p. IOL 200 has an anterior optical surface 200a facing the anterior surface 110a of the lens capsule 110, a posterior optical surface 200p facing the posterior surface 110p of the lens capsule 110, and a peripheral sealing portion 205 disposed between the anterior optical surface 200a and the posterior optical surface 200p. Due to the conformal fit of the lens, the peripheral sealing portion 205 of the IOL 200 sealingly contacts (i.e., interfaces with) lens capsule 110 (e.g., the peripheral surface 110s), thereby creating a compartment 210 that is partially or totally sealed. Compartment 210 is bounded posteriorly by the posterior surface 110p of lens capsule 110, anteriorly by the posterior optical surface 200p of IOL 200, and equatorially by sealing portion 205 (and, in some embodiments, at least a portion of peripheral surface 110s). Although FIG. 2A depicts the IOL 200 as being spaced away from the anterior surface 110s of lens capsule 110, embodiments of the invention include IOLs contacting all or a portion of the anterior surface 110s of the lens capsule and all or a portion of the posterior surface 110p of the lens capsule. In embodiments featuring liquid-filled IOLs, the contacting area may directly vary according to fill properties (e.g., fill volume, fill percentage, fill fluid viscosity, fill fluid pressure) of the IOL. Sealing portion 205 may establish a complete seal, or it may form or enable openings of posterior compartment 210. In various embodiments, the sealing portion 205 is merely the equatorial plane where the width of the lens capsule 110 and a portion of the IOL 200 come into contact to create a sealed space between the IOL and lens capsule. In other embodiments, the sealing portion 205 includes or consists essentially of a portion of the IOL 200 that is altered to favorably contact the lens capsule. This may be accomplished by, for example, adding a known thickness of silicone or other material to the interior or exterior of the sealing portion of the IOL 200. Optionally, the external sealing portion 205 of the IOL 200 may have adhesion-promotion structures such as high-friction surfaces, one or more square edges of IOL 200, or redundant sealing portions. In various embodiments, the sealing portion 205 includes or consists essentially of one or more fluid-filled reservoirs that may further be adjustable by varying the fluid within such reservoirs. By adjusting the inflation of one or more reservoirs, the sealing portion of the IOL may additionally function as a haptic to hold the IOL 200 within the lens capsule 110. In various embodiments of the invention, the IOL 200 may include one or more coupling features that provide beneficial mechanical and/or adhesive coupling to the lens capsule 110, as detailed in U. S. patent application Ser. No. 14/670,686, filed Mar. 27, 2015, the entire disclosure of which is incorporated by reference herein.

As shown in FIG. 2B, posterior compartment 210 may be partially or substantially completely filled with a fluid (typically a liquid) 215. Fluid 215 may be injected into compartment 210 through use of a surgical tool such as a blunt needle or soft silicone tip placed on a tube. In various embodiments, the tool is used to push or indent a flexible IOL 200, thereby creating a fluid pathway proximate or through sealing portion 205. In other embodiments, fluid 215 passes to posterior compartment 210 from an injection tube and the pressure of the fluid causes a separation between IOL 200 and lens capsule 110. In yet other embodiments, channels are formed, either through IOL 200 or in the periphery of IOL 200. Such channels may pass through the sealing portion 205, thereby allowing fluid continuity between posterior compartment 210 and the surrounding aqueous humor. As shown in FIG. 2B, when laser energy 220 (or other technique) is utilized to form an opening in the posterior lens capsule, the fluid 215 spaces the IOL 200 away from the posterior lens capsule, thereby preventing damage to the IOL 200.

In various embodiments of the invention, IOL 200 includes, corresponds to, or consists essentially of a fluid-filled IOL (i.e., an IOL including or consisting essentially of one or more compartments that may be filled, emptied, and refilled with a liquid for adjusting the fit and/or accommodative power of the IOL). Filling the fluid-filled IOL 200 creates a conformal fit between lens capsule 110 and lens 200. This conformal fit provides a partially or completely sealed compartment 210 to temporarily allow injection of fluid 215 between the lens capsule 110 and IOL 200. When the fluid 215 is stable or of sufficient viscosity, the fluid will remain within compartment 210 for an extended period of time. After implantation of IOL 200 and injection of fluid 215, the surgical case may be closed and the patient released. The posterior lens capsule may be opened either at the time of surgery (e.g., intraoperative), immediately after surgery, or at a later date.

Fluid 215 may remain in compartment 210 for a prolonged period of time, thereby allowing the opening of the posterior lens capsule to be performed well after the introduction of fluid 215. The total amount fluid 215 that remains in the posterior lens capsule depends on the initial amount of fluid injected, the viscosity of the fluid, the amount of sealing provided by sealing portion 205, and the ability of the fluid to diffuse through the posterior lens capsule. If it is assumed that no fluid travels through the body of IOL 200, the total amount of fluid may be estimated with the following equation:

Fluid (t)=Fluidᵢ−t(diffusion rate+leakage rate),

where Fluid(t) is the total amount of fluid in compartment 210, t is time, Fluidᵢ is the initial amount of fluid, diffusion rate is the rate of diffusion through the posterior lens capsule, and leakage rate is the rate of leakage through sealing portion 205.

The diffusion rate depends on the lens capsule thickness, permeability, and diffusion constant of fluid 215. The leakage rate through the sealing portion 205 depends on the pressure gradient between compartment 210 and the surrounding aqueous humor, the size of fluid path through sealing portion 205, and the viscosity of fluid 215. If a circular channel is formed, causing fluid to flow out, assuming laminar flow, the flow rate through the channel may be approximated using the Hagen-Poiseuille flow:

$$Leakage rate = \frac{π {Pd}^{4}}{128 μL}$$

where P is the pressure of fluid 215 relative to the surrounding aqueous, d is the diameter of the fluid path, L is the length of the fluid path, and μ is the dynamic viscosity of the fluid. Thus, higher viscosity fluids flow at a slower rate through any leakage path. In addition, in embodiments in which there is perfect sealing via sealing portion 205, then leakage depends only on diffusion.

When sealing is very good via sealing portion 205, fluid 215 may remain in compartment 210 for extended periods of time. Fluid has been demonstrated to remain in compartment 210 for more than one day, and in certain cases more than one week, and even longer than one month with appropriate sealing of a liquid-filled IOL. Sealing is often better when a fluid-filled IOL is used, as the filling amount may be tailored to the individual lens capsule 110.

In various embodiments of the invention, the fluid 215 may also be selected for its energy-dissipation properties to prevent any pockets of high energy (e.g., high temperature) caused by, e.g., laser energy. Alternatively, fluids 215 that resist cavitation may be selected if an ultrasound cutting tool is to be utilized; thus, any cavitation of the fluid will be minimal and will not damage the IOL 200 if spaced a sufficient distance.

As mentioned above, an appropriate spacing between the lens capsule 110 and the IOL 200 enables the targeting of the posterior lens capsule without damaging IOL 200. Because in various embodiments the spacing is related to the amount of fluid 215 in compartment 210, the surgical timeframe to remove the posterior lens capsule may be determined, at least in part, by the stability of the fluid 215 in compartment 210. For example, if an adequate amount of fluid 215 remains for 12 months, then the surgeon may implant the IOL and place fluid 215 in compartment 210, and the posterior lens capsule may be opened any time from the implantation period up to 12 months later.

Fluid stability may also be important when cutting an aperture in lens capsule 110. The cutting forms an opening 225, from which fluid 215 may leak out of compartment 210 while the remainder of the cutting occurs. This leakage loss of fluid 215 through opening 225 depends on the size of the hole as it is cut, pressure differential between compartment 210 and surrounding fluid, and the viscosity and cohesiveness of the filling fluid 215. Therefore, when the aperture through the posterior lens capsule is formed (via, e.g., application of laser energy), a viscous fluid 215 with a low pressure may be utilized in various embodiments of the invention, thereby enabling a slow release of the fluid 215 from compartment 210 to the surrounding environment. Slow release allows opening of the lens capsule while maintaining the integrity of the compartment 210. If a low-viscosity fluid is used, compartment 210 may collapse as the lens capsule is opened via a rapid efflux of fluid 215. Thus, in various embodiments, the viscosity of fluid 215 is nominally approximately 1 centistoke or higher, or even approximately 100 centistokes or higher. When a fluid 215 including or consisting essentially of viscoelastic is used, viscosity is often higher than 10,000 centistokes. In various embodiments, the viscosity of fluid 215 is less than approximately 700,000 centistokes. The viscosity of fluid 215 may be selected by, e.g., selecting one or combining two or more fluids such as viscoelastics, and/or by altering primarily glucose concentration and/or any protein or polypeptide composition of the fluid 215. In addition, fluid 215 should be safe to the surrounding ocular structures and preferentially absorbable by the surrounding ocular structures. In various embodiments, the fluid 215 is clear and of a similar refractive index to the aqueous humor of the eye, thus preventing any visual disturbances that may occur from the fluid 215 being inside the eye.

For clarity, an example of the above-described technique is presented. First, a compartment 210 between the posterior part of the IOL and the posterior capsule is created either at the time of IOL implantation or at a later date. The separation between the IOL and the posterior capsule may be created by injection of any fluid (e.g., water, balanced salt solution, ringer lactate, viscoelastic (e.g., sodium hyaluronate, chondroitin sulfate, and/or hydroxypropyl methycellulose), or others) or any type of gas. The fluid may either be transparent or dyed with a biocompatible solution (i.e.; steroids, trypan blue, indocyanine green). The injection of the fluid may be performed using a needle, a blunt needle, and/or a tool that provides continuous irrigation. The infusion of fluid may be provided by, for example, an infusion pump or from a bottle of balanced salt solution attached to a pole. Next, the IOL is moved anteriorly or to the side. This may be performed mechanically with a surgical instrument, injection of a fluid, or in embodiments using a fluid-filled lens, by partially or completely deflating the IOL. (For example, a fluid-filled IOL may be deflated with a tool that can aspirate the fluid through a line and/or needle. Such a system may also contain an infusion system that keeps the anterior chamber of the eye pressurized. This deflation moves the IOL out of the 3 mm center of the posterior capsule in a fashion to facilitate the laser.) Next, a laser source (Nd:YAG laser, diode laser, argon laser, excimer laser, femtosecond laser or others) or ultrasound source (ultra-high frequency) is used to rupture the posterior capsule. In cases in which the laser is not sufficient to create a rupture of the posterior capsule, an additional mechanical force such as injection of gas or liquid may be applied to cause the rupture of the posterior capsule. Finally, the IOL is re-inflated (if necessary). A sealing portion of the IOL may be used to interact with the posterior lens capsule. This system may additionally contain an aspiration feature to remove the fluid used to separate the IOL and posterior capsule (thus forming compartment 210) either during or after the rupturing of the posterior capsule. The aspiration feature may additionally remove any tissue removed during the PCO treatment.

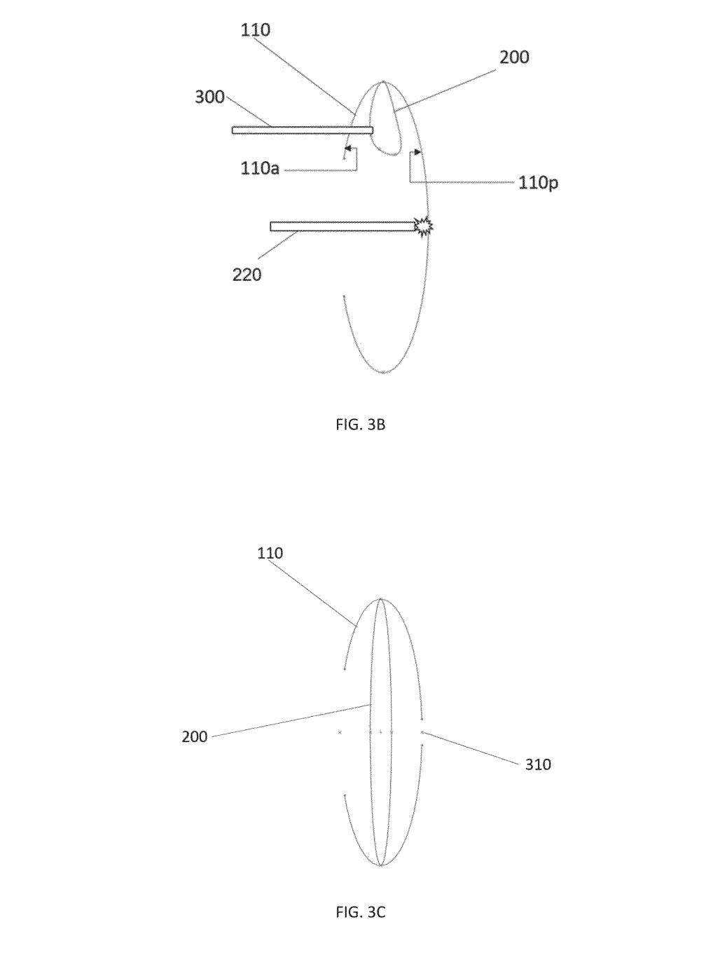

In other embodiments of the present invention, a fluid-filled (or otherwise inflatable) IOL 200 may simply be partially or fully deflated (i.e., exhausted of fluid from one or more internal compartments) before the posterior lens capsule is opened. As shown in FIGS. 3A-3C, in such embodiments, such deflation removes the IOL 200 from the path of the laser light (and from the site of the desired opening in the posterior lens capsule), thereby preventing damage to the IOL 200 during the procedure. FIG. 3A depicts a fluid-filled IOL 200 in place within the lens capsule 110 before deflation. As shown in FIG. 3B, a needle 300 is utilized to deflate the IOL 200 (by, e.g., piercing one or more surfaces of IOL 200 or interfacing with a fluid inflow/outflow port on IOL 200) to an extent sufficient to remove IOL 200 from the path of laser 220 (or other aperture-forming means) during the opening of the posterior lens capsule. The IOL 200 may be fabricated to fold into specific conformal states (e.g., saddle, roll) at different fill volumes and fill percentages that are also utilized during the implantation process to minimize the incision size in the cornea (e.g., less than 3 mm). The deflation of the IOL 200 may be accompanied by the introduction of fluid 215 behind the IOL 200, particularly in cases where the viscosity of the fluid 215 keeps all or a portion the fluid 215 in position long enough for the aperture to be opened. As shown in FIG. 3C, the IOL 200 may be reinflated (e.g., via influx of fluid through needle 300) after the opening of the aperture 310 through the posterior lens capsule.

As mentioned above, in various embodiments of the present invention, a perforation pattern (i.e., one or more openings, cuts, or perforations) may be cut or otherwise introduced into the posterior portion of the lens capsule in order to facilitate later removal thereof. While the perforation pattern is typically introduced prior to implantation of an IOL, the perforation pattern may also be introduced after implantation of the IOL (e.g., after partial or complete deflation of a fluid-filled IOL to move the IOL out of the path of the cutting implement). In various embodiments, the perforation pattern is introduced before removal of the natural lens in cataract surgery, or immediately thereafter (i.e., during the same surgical procedure or one immediately following the cataract removal).

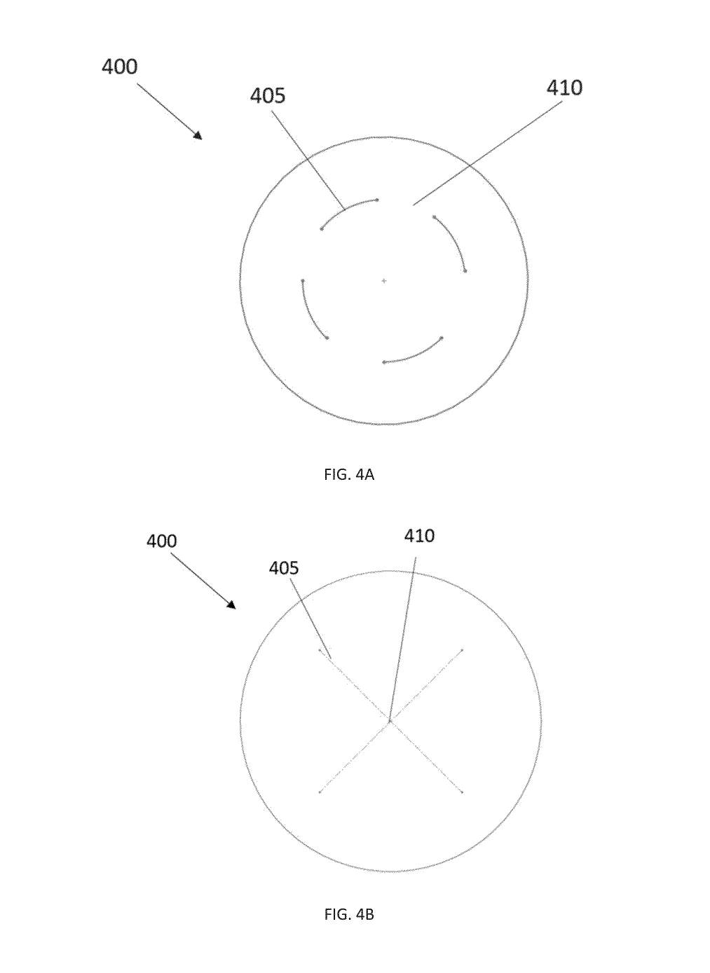

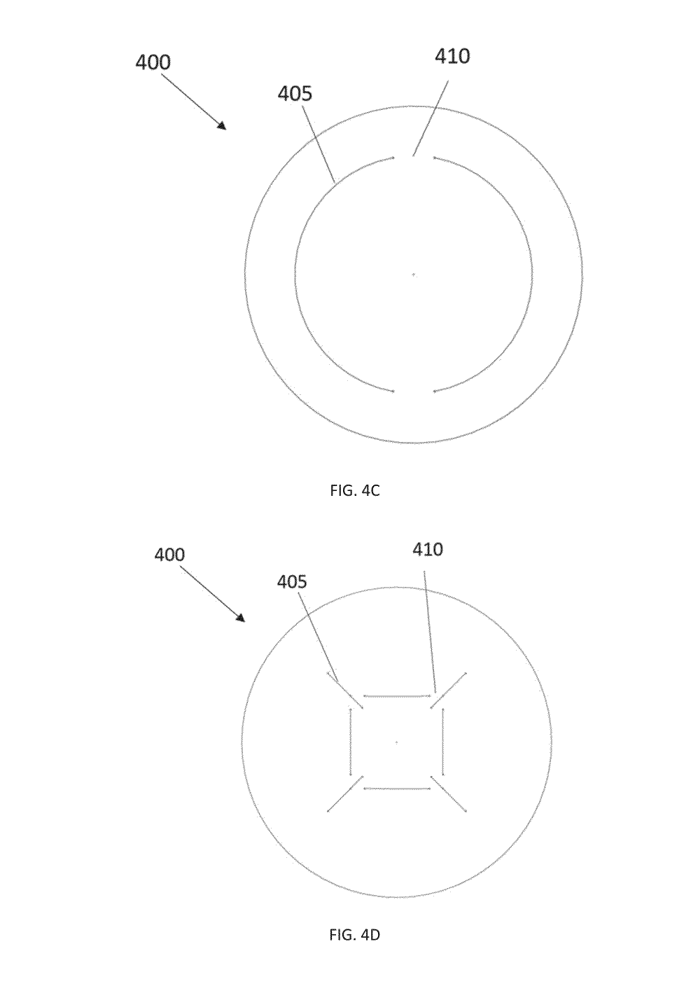

As shown in FIG. 4A, a perforation pattern in a posterior lens capsule 400 may include or consist essentially of one or more cuts 405 separated by areas 410 of intact lens capsule. The cuts 405 may extend through the entire thickness of the lens capsule or only a portion of the thickness of the lens capsule. Despite the one or more cuts 405 in the perforation pattern, in various embodiments the posterior lens capsule 400 remains structurally sound and substantially sealed to thus prevent vitreous from the posterior chamber of the eye from entering the capsule and/or the anterior chamber of the eye. However, the presence of the perforation pattern facilitates removal of a portion of the posterior lens capsule 400 after implantation of an IOL. Although the perforation pattern in FIG. 4A is depicted as circular and having four cuts 405, embodiments of the invention include perforation patterns having other shapes and including more or fewer than four cuts 405. FIGS. 4B-4D depict other exemplary perforation patterns in accordance with embodiments of the present invention.

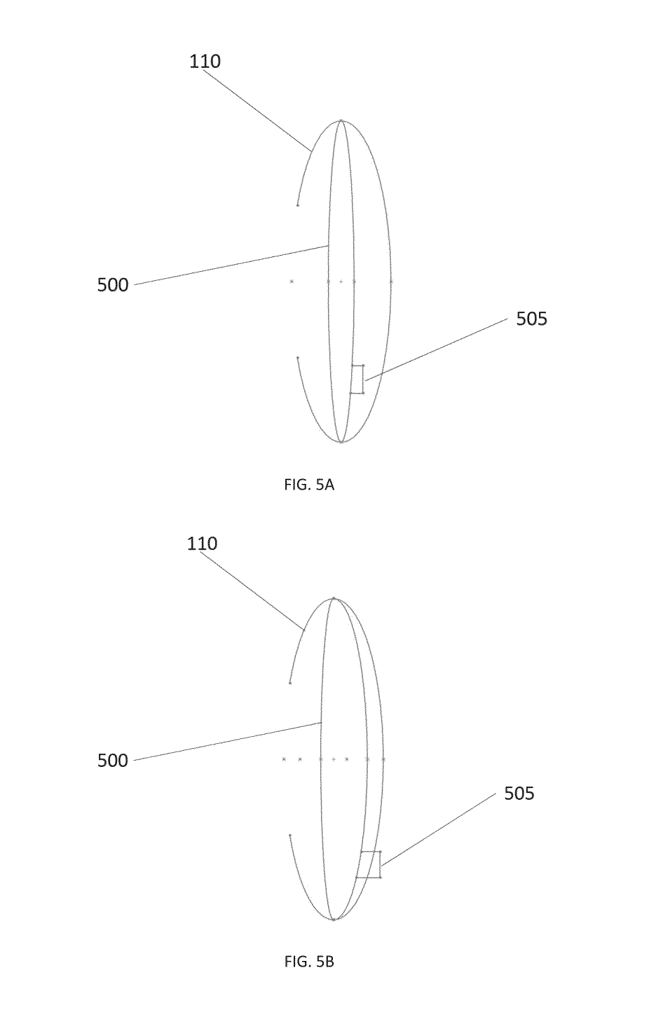

As shown in FIGS. 5A and 5B, an IOL itself may be utilized to create an aperture in the posterior lens capsule (e.g., a capsule having a perforated pattern formed therein). FIG. 5A depicts an inflatable IOL 500 disposed within the lens capsule and having an aperture-forming feature 505 thereon. The feature 505 may include or consist essentially of, for example, a protruding portion of the IOL 500, a locally abrasive (e.g., roughened) structure, a ring disposed at least partially around the periphery of IOL 500, or a shaped edge (e.g., square edge) or portion thereof of IOL 500. These features may further prevent or limit lens epithelial cell migration. FIG. 5A depicts IOL 500 is a partially or completely deflated configuration. As shown in FIG. 5B, the IOL 500 may be partially or completely inflated (i.e. filled with fluid) so as to engage feature 505 with the perforated pattern on the posterior lens capsule and break one or more of the intact areas of the pattern. In various embodiments, the IOL 500 may even be rotated to thereby apply shear stress to the perforated pattern. The action of the feature 505 (or in some embodiments, merely the surface of the IOL 500 itself) is sufficient to break one or more intact sections in the perforated pattern, opening an aperture in the posterior lens capsule.

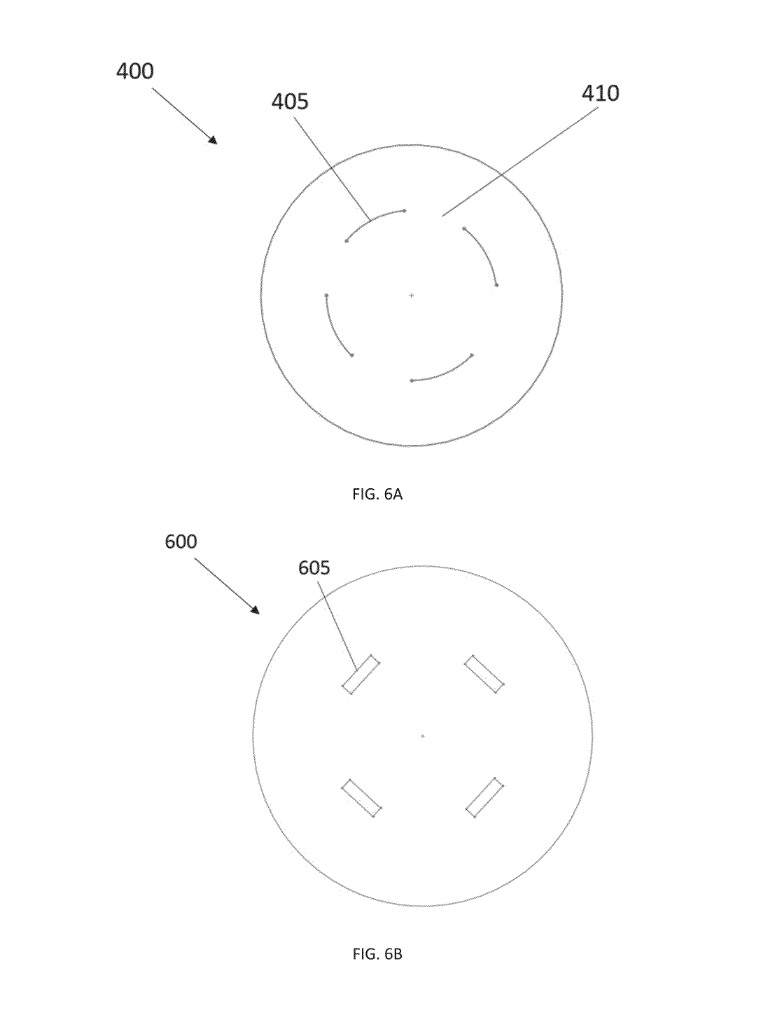

In various embodiments, an IOL contains markings (e.g., fiduciary markings) therein or thereon that align with features of a perforated pattern cut into a posterior lens capsule. (Equivalently, a perforated pattern may be cut into a posterior lens capsule, where the pattern is aligned with markings on an IOL to be implanted into the lens capsule.) The markings may additionally be used for correct placement of the IOL to achieve desired visual acuity (e.g., toric features that require a particular rotational alignment). As shown in FIGS. 6A and 6B, in an exemplary embodiment, an IOL 600 features one or more markings 605 that may be aligned with intact sections 410 cut into (or to be cut into) the posterior lens capsule 400. The markings 605 may include or consist essentially of portions of the IOL 600 that are more resistant to mechanical and/or laser-induced damage, so that the intact sections 410 may be cut, e.g., using a laser, while the IOL 600 is implanted in place in front of the perforated pattern in the posterior lens capsule. For example, the markings 605 may have thicknesses greater than that of other portions of the IOL 600, and/or they may be coated with a strengthening coating (e.g., parylene). As mentioned above, the perforated pattern is typically cut into the posterior lens capsule prior to implantation of the IOL 600. Once the IOL 600 is implanted, then an aperture may be formed in the posterior lens capsule at a desired time by, for example, focusing laser light through the markings 605 such that the intact sections 410 are removed. Such embodiments of the invention may also be utilized while the IOL is urged away from the posterior lens capsule by, e.g., introduction of fluid therebetween (as detailed above).

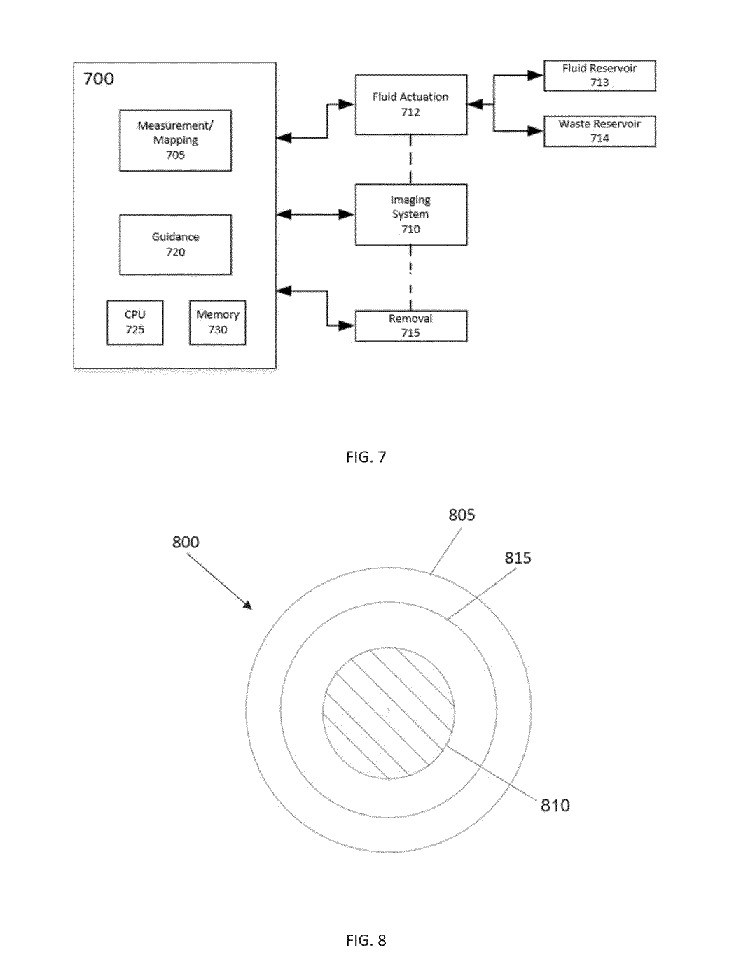

In accordance with various embodiments of the invention, apertures may be cut into posterior lens capsules via the use of an optically guided ablation system such as a laser. In various embodiments, the optically guided system is integrated with, or a portion of, an imaging system such as a microscope or an optical coherence tomography (OCT) system, and in other embodiments the optically guided system is a separate unit that interfaces with an imaging system. For example, FIG. 7 depicts an exemplary optically guided imaging and ablation system 700 in accordance with embodiments of the present invention. As shown, the system 700 includes a measurement and mapping unit 705 that, based on images acquired by an imaging system 710, (1) recognizes any fiducial markings on an implanted IOL (as described above) that signify areas of a posterior lens capsule to be ablated (using, e.g., image-recognition routines known in the art), and/or (2) determine the spacing between portions of an implanted IOL and the posterior lens capsule therebehind (using, e.g., optical or ultra-sound-based techniques to determine the relative or absolute depth of imaged features). The imaging system 710 includes optics or a detector for obtaining an image of a patient's internal ocular anatomy and a conventional (e.g., a CCD or CMOS) image sensor to digitize the image. Optical systems include, for example, microscopes, aberrometry systems, and slit-lamp illumination systems. More sophisticated optical systems may utilize OCT or a depth-sensing camera. Images of a patient's ocular anatomy can alternatively be obtained using other forms of capture system, e.g., systems based on ultrasound (such as an ultrasound biomicroscope), a scheimpflug imaging system, a tomography system, a magnetic resonance imaging system, etc.

In accordance with embodiments of the invention, the system 700 only targets areas of a posterior lens capsule for ablation if (1) they are indicated by markings on an IOL, and/or (2) the spacing between such areas of the posterior lens capsule and the IOL are greater than a threshold, thereby ensuring that the IOL is not damaged during ablation of the posterior lens capsule. For example, the threshold spacing between a region of an IOL and an area of a posterior lens capsule may be approximately 10 μm, approximately 500 μm, or even approximately 3000 μm.

The system 700 may also incorporate a fluid actuation system 712 to control the introduction of fluid between the IOL and the posterior lens capsule, thereby increasing the spacing between the IOL and the posterior lens capsule as detailed herein. The fluid actuation system 712 may include or consist essentially of, for example, a syringe, and the amount of fluid introduced behind the IOL may be monitored and controlled in real time via feedback from the imaging system 710 and the resultant spacing measured by unit 705. Fluid actuation system 712 may also be utilized to remove fluid from behind the IOL, if desired. As shown, the fluid actuation system 712 is typically connected to, or incorporates within, one or more fluid reservoirs 713 that contain the fluid to be introduced into the eye and/or into the IOL. The fluid actuation system 712 may additionally be connected to a waste reservoir 714 that is utilized to contain the fluid and other material (e.g., pieces of the posterior capsule and/or large masses of epithelial cells) removed from the IOL or eye.

As shown, the system 700 may also incorporate an ablation or cutting or removal tool 715 (e.g., a laser, ultrasound unit, mechanical cutting apparatus, etc.) for ablation or cutting or removal of portions of the posterior lens capsule identified by the unit 705. The removal tool 715 may be integrated with the imaging system 710. The system 700 may also incorporate a guidance unit 720 that provides guidance (e.g., automated guidance) of the removal tool 715 and/or the imaging system 710 based on the data acquired by the unit 705. In various embodiments, the guidance unit 720 may guide the removal tool 715 and/or the imaging system 710 based on one or more maps created by unit 705 based on the imaged data. Such maps may indicate, for example, one or more regions of a posterior lens capsule that are sufficiently spaced away from an implanted IOL such that ablation of the capsule portions will not damage the IOL. FIG. 8 depicts an exemplary map 800 of a posterior lens capsule 805 indicating, by a shaded area 810, a region of the posterior lens capsule 805 that may be targeted for ablation without damage to a nearby IOL 815. The map 800 may be generated by analysis of one or more images obtained by the imaging system 710. The path to be followed by the removal tool 715 may be determined both by the map 800, which reflects the individual patient's ocular anatomy, and a canonical cutting pattern that is selected for and/or tailored to that anatomy.

In particular, as shown in FIG. 7, the system 700 also typically includes a processor 725, for executing and/or controlling units 705, 720, and a memory 730 for storage of, e.g., maps such as map 800 and images of the interior of the patient's eye. The memory 730 may be internal and/or external to the system 700. The processor 725 directs the imaging system 710 to generate the map 800 and, if necessary or desired, to analyze the map to determine where the cuts should be made. In addition, the imaging system 710 may use the map analysis to select among a plurality of cutting patterns in order to optimize the procedure to the patient's ocular anatomy (e.g., to avoid or exploit detected points of tissue weakness). The guidance unit 720 causes the cuts to be made in accordance with the selected, optimized pattern as described above. The various components of system 700 may be implemented by computer-executable instructions, such as program modules, that are executed by a conventional computer. Generally, program modules include routines, programs, objects, components, data structures, etc. that performs particular tasks or implement particular abstract data types. Those skilled in the art will appreciate that embodiments of the invention may be practiced with various computer system configurations, including multiprocessor systems, microprocessor-based or programmable consumer electronics, minicomputers, mainframe computers, and the like. Embodiments of the invention may also be practiced in distributed computing environments wherein imaging and/or ablation are performed by remote systems that are linked through a communications network. In a distributed computing environment, program modules may be located in both local and remote computer-storage media including memory storage devices.

Any suitable programming language may be used to implement without undue experimentation the analytical functions described above. Illustratively, the programming language used may include assembly language, Ada, APL, Basic, C, C++, C*, COBOL, dBase, Forth, FORTRAN, Java, Modula-2, Pascal, Prolog, Python, REXX, and/or JavaScript for example. Further, it is not necessary that a single type of instruction or programming language be utilized in conjunction with the operation of the system and methods of embodiments of the invention. Rather, any number of different programming languages may be utilized as is necessary or desirable.

The computing environment may also include other removable/nonremovable, volatile/nonvolatile computer storage media. For example, a hard disk drive may read or write to nonremovable, nonvolatile magnetic media. A magnetic disk drive may read from or writes to a removable, nonvolatile magnetic disk, and an optical disk drive may read from or write to a removable, nonvolatile optical disk such as a CD-ROM or other optical media.

The processor that executes commands and instructions may be a general-purpose processor, but may utilize any of a wide variety of other technologies including special-purpose hardware, a microcomputer, mini-computer, mainframe computer, programmed micro-processor, micro-controller, peripheral integrated circuit element, a CSIC (Customer Specific Integrated Circuit), ASIC (Application Specific Integrated Circuit), a logic circuit, a digital signal processor, a programmable logic device such as an FPGA (Field Programmable Gate Array), PLD (Programmable Logic Device), PLA (Programmable Logic Array), RFID processor, smart chip, or any other device or arrangement of devices that is capable of implementing the steps of the processes of embodiments of the invention.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A method of treating posterior capsule opacification (PCO) in a patient's eye comprising (i) a lens capsule having a posterior surface exhibiting PCO, and (ii) an intraocular lens implanted within the lens capsule, the method comprising:
urging the intraocular lens away from the posterior surface of the lens capsule so that a distance between the intraocular lens and the lens capsule is greater than a threshold distance; and
thereafter, removing at least a portion of the posterior surface of the lens capsule.

2. The method of claim 1, wherein the threshold distance is 10 μm.

3. The method of claim 1, wherein the threshold distance is 500 μm.

4. The method of claim 1, wherein the threshold distance is 3000 μm.

5. The method of claim 1, wherein removing the at least a portion of the posterior surface of the lens capsule comprises focusing laser or ultrasound energy thereon.

6. The method of claim 1, wherein urging the intraocular lens away from the posterior surface of the lens capsule comprises introducing a fluid between the intraocular lens and the posterior surface of the lens capsule.

7. The method of claim 6, wherein a viscosity of the fluid is at least 1 centistoke.

8. The method of claim 6, wherein a viscosity of the fluid is at least 1000 centistokes.

9. The method of claim 1, wherein the intraocular lens comprises (i) a peripheral edge at least a portion of which is sealed against the lens capsule, and (ii) a posterior optical surface in opposed relation to the posterior surface of the lens capsule to form a compartment including both of the surfaces.

10. The method of claim 1, wherein (i) the intraocular lens is an inflatable intraocular lens, and (ii) urging the intraocular lens away from the posterior surface of the lens capsule comprises removing fluid from at least a portion of the intraocular lens.

11. An intraocular lens for implantation into a lens capsule of an eye, the intraocular lens comprising:
a lens portion for replacement of a natural lens of the eye; and
disposed peripherally to the lens portion, a sealing portion configured to sealingly contact the lens capsule to form a sealed compartment between the intraocular lens and a posterior surface of the lens capsule.

12. The intraocular lens of claim 11, wherein the sealing portion is pierceable and/or moveable to allow introduction of fluid between the intraocular lens and the posterior surface of the lens capsule.

13. A method of treatment of an eye comprising a lens capsule from which a natural lens has been removed, the method comprising:
forming a perforation pattern in a posterior surface of the lens capsule, the perforation pattern comprising (i) one or more perforations extending through at least a portion of a thickness of the lens capsule, and (ii) one or more intact areas proximate the one or more perforations;
after formation of the perforation pattern, implanting an intraocular lens within the lens capsule; and
after implantation of the intraocular lens, forming an aperture within the posterior surface of the lens capsule by ablating at least one of the intact areas of the perforation pattern.

14. The method of claim 13, wherein forming the perforation pattern comprises cutting the posterior surface of the lens capsule mechanically or by application of energy to the posterior surface of the lens capsule.

15. The method of claim 13, further comprising, prior to forming the aperture, urging at least a portion of the intraocular lens away from the posterior surface of the lens capsule.

16. The method of claim 15, wherein urging the at least a portion of the intraocular lens away from the posterior surface of the lens capsule comprises introducing a fluid between the intraocular lens and the posterior surface of the lens capsule.

17. The method of claim 13, wherein forming the aperture comprises urging at least a portion of the intraocular lens against at least one of the intact areas of the perforation pattern.

18. The method of claim 13, wherein the intraocular lens comprises one or more fiducial markings thereon.

19. The method of claim 18, wherein implanting the intraocular lens within the lens capsule comprises substantially aligning at least one fiducial marking with an intact area of the perforation pattern.

20. The method of claim 18, wherein, at each fiducial marking, (i) a thickness of the intraocular lens is greater than a thickness of the intraocular lens away from the fiducial marking, and/or (ii) a coating is disposed on the intraocular lens.

21. The method of claim 18, wherein ablating at least one of the intact areas of the perforation pattern comprises focusing laser or ultrasound energy through at least one of the fiducial markings.
